SEQUENCE LISTING (9 sequences)

SEQ ID NO: 1 - DNA, 12 bp, Artificial Sequence
  Synthetic Triple-forming oligonucleotide
aaaaaggaagaa

SEQ ID NO: 2 - DNA, 12 bp, Artificial Sequence
  Synthetic Triple-forming oligonucleotide
ttcttccttttt

SEQ ID NO: 3 - DNA, 17 bp, Artificial Sequence
  Synthetic Triple-forming oligonucleotide
ctttgctcttcttctcc

SEQ ID NO: 4 - DNA, 12 bp, Artificial Sequence
  Synthetic Triple-forming oligonucleotide
ctcttcttctcc

SEQ ID NO: 5 - DNA, 17 bp, Artificial Sequence
  Synthetic Triple-forming oligonucleotide
ggagaagaagagtaaag

SEQ ID NO: 6 - DNA, 12 bp, Artificial Sequence
  Synthetic Triple-forming oligonucleotide
ggagaagaagag

SEQ ID NO: 7 - DNA, 20 bp, Artificial Sequence
  Synthetic peptide nucleioc acids
ntnttnttnttcttcttctc

SEQ ID NO: 8 - DNA, 20 bp, Artificial Sequence
  Synthetic peptide nucleioc acids
ntnttnttnttcttcttctc

SEQ ID NO: 9 - DNA, 10 bp, Artificial Sequence
  Synthetic peptide nucleioc acids
cttgtcatgg

(19) United States
(12) Patent Application Publication
(10) Pub. No.: US 2010/0172882 A1
(43) Pub. Date: Jul. 8, 2010

(54) COMPOSITIONS AND METHODS FOR TARGETED INACTIVATION OF HIV CELL SURFACE RECEPTORS

(76) Inventors: Peter M. Glazer, Guilford, CT (US); Ranjit Bindra, New York, NY (US); Erica B. Schleifman, New Haven, CT (US)

Correspondence Address: Pabst Patent Group LLP, 1545 PEACHTREE STREET NE, SUITE 320, ATLANTA, GA, 30309, US

(21) Appl. No.: 12/522,804

(22) PCT Filed: Jan. 11, 2008
(86) PCT No.: PCT/US08/50920

§ 371 Date: Mar. 2, 2010

Related U.S. Application Data
(60) Provisional application No. 60/880,232, filed on Jan. 11, 2007.

(51) Int. Cl. A61K 35/12; C12N 15/87; A61K 38/16; C12N 5/00; A61P 31/18

(52) U.S. Cl. 424/93.7; 435/463; 514/8; 435/325

(57) ABSTRACT

Compositions for targeted mutagenesis of cell surface receptors for HIV and methods of their use are provided herein. The compositions include triplex-forming molecules that bind to duplex DNA in a sequence specific manner at target sites to form triple-stranded structures. The triplex-forming molecules can be triplex-forming oligonucleotides (TFOs) or peptide nucleic acids (PNAs). The triplex-forming molecules are useful to induce site-specific homologous recombination in mammalian cells when used in combination with donor oligonucleotides. The triplex-forming molecules target sites within or adjacent to genes that encodes cell surface receptors for human immunodeficiency virus (HIV). This binding stimulates homologous recombination of a donor oligonucleotide to cause mutations in HIV cell surface receptor genes that result in one or more deficiencies in the ability of the encoded receptor to bind to HIV and allow its transport into the cell. Methods for ex vivo and in vivo prophylaxis and therapy of HIV infection using the disclosed compositions are also provided.

### CROSS-REFERENCE TO RELATED APPLICATIONS

This application claims priority to U.S. Ser. No. 60/880,232 entitled “Targeted inactivation of the CCR5 gene using PNAs as an anti-HIV therapy”, filed Jan. 11, 2007.

### GOVERNMENT SUPPORT

This invention was made with government support awarded by the National Institutes of Health under Grant Number NIH R01CA64186. The United States government has certain rights in this invention.

### FIELD OF THE INVENTION

The present disclosure generally relates to the field of compositions that bind to DNA encoding cell surface receptors for HIV and methods of using these compositions.

### BACKGROUND OF THE INVENTION

HIV-1 is a member of the Retroviridae family belonging to the genus lentiviruses. The Retroviridae are enveloped viruses containing two positive sense RNA strands that are converted into dsDNA by the highly error-prone viral reverse transcriptase enzyme generating isolate diversity by both point mutation and intergenomic recombination. HIV-1 isolates fall into three groups: M (Major/Main), N (Non-M, Non-O/New) and O (Outlier) of which, as implied, group M is most common. Group M is subdivided into several subtypes or clades (A-D, F-H, J and K), of which B is most common in the Western world, whilst C is the predominant subtype found primarily in India, China and sub-Saharan Africa. The remaining subtypes, as well as HIV-1 variants with characteristics of several different subtypes, so-called circulating recombinant forms (CRFs), are dispersed throughout Africa and other parts of the world.

HIV-1 contains the exterior envelope glycoprotein, gp120, and the transmembrane glycoprotein, gp41. These proteins are generated by cleavage of a heavily glycosylated precursor protein, gp160, by furin-like enzymes during transport through the Golgi apparatus. Once transported to the cell surface, trimeric gp120/gp41 envelope glycoprotein spikes are incorporated into budding virus for release of new HIV-1 particles. Each new infectious cycle is initiated when the external envelope glycoprotein gp120 binds the primary receptor, CD4, which is embedded in the plasma membrane on the surface of potential targets cells. Interaction of gp120 with CD4 is followed by a series of conformational changes in Env resulting in exposure of a transient binding site that allows the spike to interact with its co-receptor, usually CCR5 or CXCR4. This in turn promotes additional conformational changes that allow gp41 to insert its fusion peptide into the target cell membrane to form a prehairpin structure, which then collapses into an energetically stable six-helix bundle structure, driving virus-to-cell membrane fusion and entry of the HIV-1 core into the target cell. This sequence of event occurs at the plasma membrane at neutral pH.

Entry inhibitors have recently emerged as a new class of HIV therapeutics which could potentially change the treatment paradigm. These drugs block cell surface receptors required for HIV entry into T-cells, such as the protein encoded by the CCR5 gene. The CCR5 chemokine receptor is a major co-receptor for R5-tropic HIV-1 strains, which are responsible for most cases of initial, acute HIV infection. Individuals who possess a homozygous inactivating mutation (referred to as the Delta32 mutation) in the CCR5 gene are almost completely resistant to infection by R5-tropic HIV-1 strains, with no other significant adverse consequences. With over 40 million people currently living with AIDS, industry analysts estimate that a successful therapy targeting CCR5 will generate sales of $500-700 MM per year. A number of pharmaceutical companies are currently trying to develop entry-inhibitor drugs to block the receptor protein, although progress has been hindered by toxicity, efficacy and drug resistance.

Since the initial observation of triple-stranded DNA many years ago by Felsenfeld et al., J. Am. Chem. Soc. 79:2023 (1957), oligonucleotide-directed triple helix formation has emerged as a valuable tool in molecular biology. Current knowledge suggests that oligonucleotides can bind as third strands of DNA in a sequence specific manner in the major groove in polypurine/polypyrimidine stretches in duplex DNA. In one motif, a polypyrimidine oligonucleotide binds in a direction parallel to the purine strand in the duplex, as described by Moser and Dervan, Science 238:645 (1987), Praseuth et al., Proc. Natl. Acad. Sci. USA 85:1349 (1988), and Mergny et al., Biochemistry 30:9791 (1991). In the alternate purine motif, a polypurine strand binds anti-parallel to the purine strand, as described by Beal and Dervan, Science 251:1360 (1991). The specificity of triplex formation arises from base triplets (AAT and GGC in the purine motif) formed by hydrogen bonding; mismatches destabilize the triple helix, as described by Mergny et al., Biochemistry 30:9791 (1991) and Beal and Dervan, Nuc. Acids Res. 11:2773 (1992).

Triplex forming oligonucleotides (TFOs) are useful for several molecular biology techniques. For example, triplex forming oligonucleotides designed to bind to sites in gene promoters have been used to block DNA binding proteins and to block transcription both in vitro and in vivo. (Maher et al., Science 245:725 (1989), Orson et al., Nucleic Acids Res. 19:3435 (1991), Postal et al., Proc. Natl. Acad. Sci. USA 88:8227 (1991), Cooney et al., Science 241:456 (1988), Young et al., Proc. Natl. Acad. Sci. USA 88:10023 (1991), Maher et al., Biochemistry 31:70 (1992), Duval-Valentin et al., Proc. Natl. Acad. Sci. USA 89:504 (1992), Blume et al., Nucleic Acids Res. 20:1777 (1992), Durland et al., Biochemistry 30:9246 (1991), Grigoriev et al., J. of Biological Chem. 267:3389 (1992), and Takasugi et al., Proc. Natl. Acad. Sci. USA 88:5602 (1991)). Site specific cleavage of DNA has been achieved by using triplex forming oligonucleotides linked to reactive moieties such as EDTA-Fe(II) or by using triplex forming oligonucleotides in conjunction with DNA modifying enzymes (Perrouault et al., Nature 344:358 (1990), Francois et al., Proc. Natl. Acad. Set USA 86:9702 (1989), Lin et al., Biochemistry 28:1054 (1989), Pei et al., Proc. Natl. Acad. Sci. USA 87:9858 (1990), Strobel et al., Science 254:1639 (1991), and Posvic and Dervan, J. Am. Chem. Soc. 112:9428 (1992)). Sequence specific DNA purification using triplex affinity capture has also been demonstrated. (Ito et al., Proc. Natl. Acad. Sci. USA 89:495 (1992)). Triplex forming oligonucleotides linked to intercalating agents such as acridine, or to cross-linking agents, such as p-azidophenacyl and psoralen, have been utilized, but only to enhance the stability of triplex binding. (Praseuth et al., Proc. Natl. Acad. Sci. USA 85:1349 (1988), Grigoriev et al., J. of Biological Chem. 267:3389 (1992), Takasugi et al., Proc. Natl. Acad. Sci. USA 88:5602 (1991).

Gene therapy can be defined by the methods used to introduce heterologous DNA into a host cell or by the methods used to alter the expression of endogenous genes within a cell. As such, gene therapy methods can be used to alter the phenotype and/or genotype of a cell.

Targeted modification of the genome by gene replacement is of value as a research tool and in gene therapy. However, while facile methods exist to introduce new genes into mammalian cells, the frequency of homologous integration is limited (Hanson et al., (1995) Mol. Cell. Biol. 15(1), 45-51), and isolation of cells with site-specific gene insertion typically requires a selection procedure (Capecchi, M. R., (1989) Science 244(4910), 1288-1292). Site-specific DNA damage in the form of double-strand breaks produced by rare cutting endonucleases can promote homologous recombination at chromosomal loci in several cell systems, but this approach requires the prior insertion of the recognition sequence into the locus.

Methods which alter the genotype of a cell typically rely on the introduction into the cell of an entire replacement copy of a defective gene, a heterologous gene, or a small nucleic acid molecule such as an oligonucleotide, to treat human, animal and plant genetic disorders. The introduced gene or nucleic acid molecule, via genetic recombination, replaces the endogenous gene. This approach requires complex delivery systems to introduce the replacement gene into the cell, such as genetically engineered viruses, or viral vectors.

Alternatively, gene therapy methods can be used to alter the expression of an endogenous gene. One example of this type of method is antisense therapy. In antisense therapy, a nucleic acid molecule is introduced into a cell, the nucleic acid molecule being of a specific nucleic acid sequence so as to hybridize or bind to the mRNA encoding a specific protein. The binding of the antisense molecule to an mRNA species decreases the efficiency and rate of translation of the mRNA.

Gene therapy is being used on an experimental basis to treat well known genetic disorders of humans such as retinoblastoma, cystic fibrosis, and globinopathies such as sickle cell anemia. However, in vivo efficiency is low due to the limited number of recombination events actually resulting in replacement of the defective gene.

Compositions and methods for targeted mutagenesis of genes encoding cell surface receptors for HIV would be useful as a means of gene therapy for use in ex vivo and in vivo prophylactic and therapeutic applications. Such compositions and methods would also be useful for generating cells with a spectrum of mutations in genes encoding cell surface receptors for HIV for use as research tools.

Therefore it is an object of the invention to provide compositions and methods of use thereof for in vivo and ex vivo targeted recombination at sites of or adjacent to genes that encode cell surface receptors for HIV.

It is a further object of the present invention to provide compositions and methods of use thereof that induce targeted mutagenesis at sites of or adjacent to genes that encode cell surface receptors for HIV.

It is a further object of the invention to provide cells that contain mutations at sites of or adjacent to genes that encode cell surface receptors for HIV.

It is a further object of the present invention to provide compositions and methods for treating or preventing HIV infection by gene therapy without the need for a viral vector.

It is a further object of the invention to provide compositions and methods for treating or preventing HIV infection by ex vivo gene therapy.

It is a further object of the invention to provide compositions and methods for treating or preventing HIV infection by in vivo gene therapy.

### SUMMARY OF THE INVENTION

Compositions for targeted mutagenesis of cell surface receptors for HIV and methods of their use are provided herein. The compositions include triplex-forming molecules that bind to duplex DNA in a sequence specific manner at target sites to fowl triple-stranded structures.

The target site is within or adjacent to a gene that encodes a cell surface receptor for human immunodeficiency virus (HIV). The HIV cell surface receptor can be a chemokine receptor, including CXCR4, CCR5, CCR2b, CCR3 and CCR1. The target site can be within the coding region of the gene. The target sequence is preferably within or adjacent to a portion of the HIV cell surface receptor gene that important to its function in allowing HIV entry into cells, such as nucleotides or nucleotide sequences involved in efficient expression of the receptor, transport of the receptor to the cell surface, stability of the receptor, viral binding by the receptor, or endocytosis of the receptor. In one embodiment, the target site for the triplex-forming molecule is within or adjacent to the human CCR5 gene. In a preferred embodiment, the target site encompasses or is adjacent to the site of a naturally occurring nonsense mutation referred to as the Δ32 mutation.

The triplex-forming molecules can be triplex-forming oligonucleotides (TFOs). TFOs are single-stranded oligonucleotides between about 7 and about 40 nucleotides in length. TFOs bind to target sites containing polypurine, homopurine, polypyrimidine or homopyrimidine base compositions within a major grove of duplex DNA. TFOs can contain chemical modifications to their nucleotide constituents, including chemical modifications of their heterocyclic bases, sugar moieties or phosphate moieties. These modifications can increase the biding affinity of the TFO for the target site or the stability of the fowled triplex.

The triplex-forming molecules can also be peptide nucleic acids (PNAs). Highly stable PNA:DNA:PNA triplex structures can be formed from strand invasion of a duplex DNA with two PNA strands. The two PNA strands can be linked together to form a bis-PNA molecule. PNAs also bind to target sites with polypurine or homopurine sequences, but can do so at shorter target sequences relative to TFOs, and with greater stability.

The triplex-forming molecules are useful to induce site-specific homologous recombination in mammalian cells when used in combination with donor oligonucleotides. Donor oligonucleotides can be tethered to triplex-forming molecules or can be separate from the triplex-forming molecules. The donor oligonucleotides can contain at least one nucleotide mutation, insertion or deletion relative to the target duplex DNA. Triplex-forming molecules can be used in conjunction with donor oligonucleotides to cause mutations in HIV cell surface receptor genes that result in one or more deficiencies in the ability of the encoded receptor to bind to HIV and allow its transport into the cell. Suitable mutations are those that result in a decrease in the expression of a cell surface HIV receptor, its transport to the cell surface, its stability, its ability to bind to HIV, or its endocytosis.

Also provided are cell lines generated by contacting cells with triplex-forming molecules and donor oligonucleotides that contain at least one mutation in a cell surface receptor for HIV. The cells are preferably hematopoietic in origin. Useful hematopoietic cells include T cells and hematopoietic stem cells including CD34⁺ cells. The cell lines can be used for the screening and development of other HIV therapeutic agents, including other agents that reduce or inhibit the entry of HIV into cells.

Also provided are prophylactic and therapeutic methods for treating subjects with or at risk of developing an HIV infection using the compositions disclosed herein. The methods can be used to prevent infection of an individual with HIV or to reduce the viral load of an individual already infected with HIV. In one embodiment, ex vivo therapy using the compositions disclosed herein is used for treatment or prevention of HIV infection. These methods include isolating target cells, contacting the target cells ex vivo with triplex-forming molecules and donor oligonucleotides to cause targeted mutagenesis of HIV cell surface receptor genes, expanding the modified cells in culture, and administering the modified cells to the subject in need thereof. The cells can be isolated from the subject to be treated or can be isolated from a syngenic or allogenic host. The cells can be hematopoietic stepm cells and are preferably CD34⁺ cells. In another embodiment, the modified cells are differentiated in ex vivo culture and expanded in large numbers prior to administration to the subject. The cells are preferably differentiated into CD4⁺T cells. Methods for treating or preventing HIV infection by administering the compositions disclosed herein are also provided.

### BRIEF DESCRIPTION OF THE DRAWINGS

FIG. 1 is a bar graph showing the relative mutation rate of the CCR5 gene in THP-1 cells transfected with either no DNA, donor oligonucleotide alone, or donor oligonucleotide in combination with a peptide nucleic acid that binds to the CCR5 gene. Results were obtained using allele-specific PCR forty-eight hours after transfection and are expressed as the relative mutation rate. Data were normalized relative to results using gene specific primers to CCR5.

### DETAILED DESCRIPTION OF THE INVENTION

### I. Compositions that Bind to Double-Stranded DNA Encoding Cell Surface Receptors for HIV

Disclosed herein are compositions containing molecules, referred to as “triplex-forming molecules”, that bind to duplex DNA in a sequence-specific manner to form a triple-stranded structure. The triplex-forming molecules can be used to induce site-specific homologous recombination in mammalian cells when combined with donor DNA molecules. The donor DNA molecules can contain mutated nucleic acids relative to the target DNA sequence. This is useful to activate, inactivate, or otherwise alter the function of a polypeptide or protein encoded by the targeted duplex DNA. Triplex-forming molecules include triplex-forming oligonucleotides and peptide nucleic acids.

A. Genes to be Targeted with Triplex-Forming Molecule

The predetermined region that the triplex-forming molecules bind to is referred to herein as the “target sequence”, “target region”, or “target site”. The target sequence for the triplex-forming molecules disclosed herein is within or adjacent to a human gene that encodes a cell surface receptor for human immunodeficiency virus (HIV). Preferably, the target sequence of the triplex-forming molecule is within or is adjacent to a portion of a HIV receptor gene important to its function in HIV entry into cells, such as sequences that are involved in efficient expression of the receptor, transport of the receptor to the cell surface, stability of the receptor, viral binding by the receptor, or endocytosis of the receptor. Target sequences can be within the coding DNA sequence of the gene or within introns. Target sequences can also be within DNA sequences which regulate expression of the target gene, including promoter or enhancer sequences.

The target sequence can be within or adjacent to any gene encoding a cell surface receptor that facilitates entry of HIV into cells. The molecular mechanism of HIV entry into cells involves specific interactions between the viral envelope glycoproteins (env) and two target cell proteins, CD4 and the chemokine receptors. HIV cell tropism is determined by the specificity of the env for a particular chemokine receptor, a 7 transmembrane-spanning, G protein-coupled receptor (Steinberger, et al., Proc. Natl. Acad. Sci. USA. 97: 805-10 (2000)). The two major families of chemokine receptors are the CXC chemokine receptors and the CC chemokine receptors (CCR) so named for their binding of CXC and CC chemokines, respectively. While CXC chemokine receptors traditionally have been associated with acute inflammatory responses, the CCRs are mostly expressed on cell types found in connection with chronic inflammation and T-cell-mediated inflammatory reactions: eosinophils, basophils, monocytes, macrophages, dendritic cells, and T cells (Nansen, et al. 2002, Blood 99:4). In one embodiment embodiment, the target sequence is within or adjacent to the human genes encoding chemokine receptors, including, but not limited to, CXCR4, CCR5, CCR2b, CCR3, and CCR1.

In a preferred embodiment, the target sequence is within or adjacent to the human CCR5 gene. The CCR5 chemokine receptor is the major co-receptor for R5-tropic HIV strains, which are responsible for most cases of initial, acute HIV infection. Individuals who possess a homozygous inactivating mutation, referred to as the Δ32 mutation, in the CCR5 gene are almost completely resistant to infection by R5-tropic HIV-1 strains. The Δ32 mutation produces a 32 base pair deletion in the CCR5 coding region.

Another naturally occurring mutation in the CCR5 gene is the m303 mutation, characterized by an open reading frame single T to A base pair transversion at nucleotide 303 which indicates a cysteine to stop codon change in the first extracellular loop of the chemokine receptor protein at amino acid 101 (C101X) (Carrington et al. 1997). Mutagenesis assays have not detected the expression of the m303 co-receptor on the surface of CCR5 null transfected cells which were found to be non-susceptible to HIV-1 R5-isolates in infection assays (Blanpain, et al. (2000).

Individuals having the homozygous Δ32 inactivating mutation in the CCR5 gene display no significant adverse phenotypes, suggesting that this gene is largely dispensible for normal human health. This makes the CCR5 gene a particularly attractive target for targeted mutagenesis using the triplex-forming molecules disclosed herein. The gene for human CCR5 is known in the art and is provided at GENBANK accession number NM_{—}000579. The coding region of the human CCR5 gene is provided by nucleotides 358 to 1416 of GENBANK accession number NM_{—}000579.

B. Triplex-Forming Oligonucleotides (TFOs)

In one embodiment, the triplex-forming molecules are triplex-forming oligonucleotides. Triplex-forming oligonucleotides (TFOs) are defined as oligonucleotides which bind as third strands to duplex DNA in a sequence specific manner. The oligonucleotides are synthetic or isolated nucleic acid molecules which selectively bind to or hybridize with a predetermined target sequence, target region, or target site within or adjacent to a human gene encoding a cell surface HIV receptor so as to form a triple-stranded structure.

Preferably, the oligonucleotide is a single-stranded nucleic acid molecule between 7 and 40 nucleotides in length, most preferably 10 to 20 nucleotides in length for in vitro mutagenesis and 20 to 30 nucleotides in length for in vivo mutagenesis. The base composition may be homopurine or homopyrimidine. Alternatively, the base composition may be polypurine or polypyrimidine. However, other compositions are also useful.

The oligonucleotides are preferably generated using known DNA synthesis procedures. In one embodiment, oligonucleotides are generated synthetically. Oligonucleotides can also be chemically modified using standard methods that are well known in the art.

The nucleotide sequence of the oligonucleotides is selected based on the sequence of the target sequence, the physical constraints imposed by the need to achieve binding of the oligonucleotide within the major groove of the target region, and the need to have a low dissociation constant (K_{d}) for the oligonucleotide/target sequence. The oligonucleotides will have a base composition which is conducive to triple-helix formation and will be generated based on one of the known structural motifs for third strand binding. The most stable complexes are formed on polypurine:polypyrimidine elements, which are relatively abundant in mammalian genomes. Triplex formation by TFOs can occur with the third strand oriented either parallel or anti-parallel to the purine strand of the duplex. In the anti-parallel, purine motif, the triplets are G.G:C and A.A:T, whereas in the parallel pyrimidine motif, the canonical triplets are C⁺.G:C and T.A:T. The triplex structures are stabilized by two Hoogsteen hydrogen bonds between the bases in the TFO strand and the purine strand in the duplex. A review of base compositions for third strand binding oligonucleotides is provided in U.S. Pat. No. 5,422,251.

Preferably, the oligonucleotide binds to or hybridizes to the target sequence under conditions of high stringency and specificity. Most preferably, the oligonucleotides bind in a sequence-specific manner within the major groove of duplex DNA. Reaction conditions for in vitro triple helix formation of an oligonucleotide probe or primer to a nucleic acid sequence vary from oligonucleotide to oligonucleotide, depending on factors such as oligonucleotide length, the number of G:C and A:T base pairs, and the composition of the buffer utilized in the hybridization reaction. An oligonucleotide substantially complementary, based on the third strand binding code, to the target region of the double-stranded nucleic acid molecule is preferred.

As used herein, an oligonucleotide is said to be substantially complementary to a target region when the oligonucleotide has a heterocyclic base composition which allows for the formation of a triple-helix with the target region. As such, an oligonucleotide is substantially complementary to a target region even when there are non-complementary bases present in the oligonucleotide. As stated above, there are a variety of structural motifs available which can be used to determine the nucleotide sequence of a substantially complementary oligonucleotide.

In one embodiment, the target region is a polypurine site within or adjacent to a gene encoding a chemokine receptor including CXCR4, CCR5, CCR2b, CCR3, and CCR1. In a preferred embodiment, the target region is a polypurine or homopurine site within the coding region of the human CCR5 gene. Three homopurine sites in the coding region of the CCR5 gene that are especially useful as target sites for triplex-forming molecules are from positions 509-518, 679-690 and 900-908 relative to the ATG start codon.

The homopurine site from 679-690 partially encompasses the site of the nonsense mutation created by the Δ32 mutation. TFOs that bind to this target site are particularly useful. Representative TFOs that an be used to bind to the CCR5 gene include, but are not limited to, the following sequences:

| | | |
|---|---|---|
| 5′-AAAAAGGAAGAA-3′ | (232-243) | (SEQ ID NO: 1) |
| 3′-TTTTTCCTTCTT-5′ | (232-243) | (SEQ ID NO: 2) |
| 5′-CTTTGCTCTTCTTCTCC-3′ | (674-690) | (SEQ ID NO: 3) |
| 5′-CTCTTCTTCTCC-3′ | (679-690) | (SEQ ID NO: 4) |
| 3′-GAAATGAGAAGAAGAGG-5′ | (674-690) | (SEQ ID NO: 5) |
| 3′-GAGAAGAAGAGG-5′ | (679-690) | (SEQ ID NO: 6) |

The numbers in parenthesis indicate the location of the CCR5 gene relative to the start ATG that the indicated TFOs bind to.

Chemical Modifications

As used herein, an “oligonucleotide” or a “polynucleotide” is a nucleic acid polymer comprising a plurality of nucleotide subunits of defined base sequence. Oligonucleotides comprise a chain of nucleotides which are linked to one another by phosphate ester linkages. Each nucleotide typically comprises a heterocyclic base (nucleic acid base), a sugar moiety attached to the heterocyclic base, and a phosphate moiety which esterifies a hydroxyl function of the sugar moiety. The principal naturally-occurring nucleotides comprise uracil, thymine, cytosine, adenine and guanine as the heterocyclic bases, and ribose or deoxyribose as the sugar moiety.

Triplex-forming oligonucleotides can include chemical modifications to their nucleotide constituents. Modified bases and base analogues, modified sugars and sugar analogues and/or phosphate analogues and modified phosphate moieties, known in the art, are also suitable for use in triplex-forming oligonucleotides. Under physiologic conditions, potassium levels are high, magnesium levels are low, and pH is neutral. These conditions are generally unfavorable to allow for effective binding of TFOs to duplex DNA. For example, high potassium promotes guanine (G)-quartet formation, which inhibits the activity of G-rich purine motif TFOs. Also, magnesium, which is present at low concentrations under physiologic conditions, supports third-strand binding by charge neutralization. Finally, neutral pH disfavors cytosine protonation, which is needed for pyrimidine motif third-strand binding. Target sequences with adjacent cytosines are particularly problematic. Triplex stability is greatly compromised by runs of cytosines, thought to be due to repulsion between the positive charge resulting from the N³ protonation or perhaps because of competition for protons by the adjacent cytosines.

Chemical modification of nucleotides comprising TFOs may be useful to increase binding affinity of TFOs and/or triplex stability under physiologic conditions. Modified nucleotides may comprise one or more of the nucleotides which comprise a triplex-forming oligonucleotide. As used herein “modified nucleotide” or “chemically modified nucleotide” defines a nucleotide that has a chemical modification of one or more of the hetrocyclic base, sugar moiety or phosphate moiety constituents. Preferably, modified oligonucleotides in TFOs are able to form Hoogsteen and/or reverse Hoogsteen base pairs with bases of the target sequence. More preferably, modified oligonucleotides increase the binding affinity of the TFO to the target duplex DNA, or the stability of the formed triplex.

Chemical modifications of hetrocyclic bases or heterocyclic base analogs may be effective to increase the binding affinity of a nucleotide or its stability in a triplex. Chemically-modified heterocyclic bases include, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives. Substitution of 5-methylcytosine or pseudoisocytosine for cytosine in TFOs helps to stabilize triplex formation at neutral pH, especially in TFOs with isolated cytosines. This is because the positive charge partially reduces the negative charge repulsion between the TFO and the target duplex. Substitutions of 2′-O-methylpseudocytidine for cytidine are especially useful to stabilize triplexes formed by TFOs and target duplexes when the target sequence contains adjacent cytidines.

Triplex-forming oligonucleotides may also contain nucleotides with modified sugar moieties or sugar moiety analogs. Sugar moiety modifications include, but are not limited to, 2′-O-aminoethoxy, 2′-O-amonioethyl (2′-OAE), 2′-O-methoxy, 2′-O-methyl, 2-guanidoethyl (2′-OGE), 2′-O,4′-C-methylene (LNA), 2′-O-(methoxyethyl) (2′-OME) and 2% O—(N-(methyl)acetamido) (2′-OMA). 2′-O-aminoethyl sugar moiety substitutions are especially preferred because they are protonated at neutral pH and thus suppress the charge repulsion between the TFO and the target duplex. This modification stabilizes the C3′-endo conformation of the ribose or dexyribose and also forms a bridge with the i-1 phosphate in the purine strand of the duplex.

Modifications to the phosphate backbone of triplex-forming oligonucleotides may also increase the binding affinity of TFOs or stabilize the triplex formed between the TFO and the target duplex. Cationic modifications, including, but not limited to, diethyl-ethylenediamide (DEED) or dimethyl-aminopropylamine (DMAP) may be especially useful due to decrease electrostatic repulsion between TFO and duplex target phosphates.

Modifications of the phosphate backbone may also include the substitution of a sulfur atom for one of the non-bridging oxygens in the phosphodiester linkage. This substitution creates a phosphorothioate internucleoside linkage in place of the phosphodiester linkage. Oligonucleotides containing phosphorothioate internucleoside linkages have been shown to be more stable in vivo.

Oligonucleotides may further be modified to be end capped to prevent degradation using a 3′ propylamine group. Procedures for 3′ or 5′ capping oligonucleotides are well known in the art.

C. Peptide Nucleic Acids

In another embodiment, the triplex-forming molecules are peptide nucleic acids (PNAs). Peptide nucleic acids are molecules in which the phosphate backbone backbone of oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that is similar to oligonucleotides, but are achiral and neutrally charged molecules, Peptide nucleic acids are comprised of peptide nucleic acid monomers. The heterocyclic bases can be any of the standard bases (uracil, thymine, cytosine, adenine and guanine) or any of the modified heterocyclic bases described above with reference to use in triplex-forming oligonucleotides.

PNAs can bind to DNA via Watson-Crick hydrogen bonds, but with binding affinities significantly higher than those of corresponding oligonucleotides. The neutral backbone of PNAs decreases electrostatic repulsion between the PNA and target DNA phosphates. Under in vitro or in vivo conditions that promote opening of the duplex DNA, PNAs can mediate strand invasion of duplex DNA resulting in displacement of one DNA strand to form a D-loop.

Highly stable triplex PNA:DNA:PNA structures can be formed from a homopurine DNA strand and two PNA strands. The two PNA strands may be linked together by a linker of sufficient flexibility to form a bis-PNA molecule that forms a triplex “clamp” with one of the strands of the target duplex while displacing the other strand of the duplex target. In this structure, one strand forms Watson-Crick base pairs with the DNA strand in the anti-parallel orientation, whereas the other strand forms Hoogsteen base pairs to the homopurine strand in the DNA-PNA duplex. Although, as with triplex-forming oligonucleotides, a homopurine strand is needed to allow formation of a stable PNA/DNA/PNA triplex, PNA clamps can form at shorter homopurine sequences than those required by triplex-forming oligonucleotides and also do so with greater stability.

Suitable molecules for use in linkers of bis-PNA molecules include, but are not limited to 8-amino-3,6-dioxaoctanoic acid, referred to as an O-linker, and 6-aminohexanoic acid. Poly(ethylene) glycol monomers can also be used in bis-PNA linkers. A bis-PNA linker can contain multiple linker molecule monomers in any combination.

PNAs can also include other positively charged moieties to increase the solubility of the PNA and increase the affinity of the PNA for duplex DNA. Commonly used positively charged moieties include the amino acids lysine and arginine, although other positively charged moieties may also be useful. Lysine and arginine residues can be added to a bis-PNA linker or can be added to the carboxy terminus of a PNA strand.

In one embodiment, the target region for binding by a PNA is a polypurine site within or adjacent to a gene encoding a chemokine receptor including CXCR4, CCR5, CCR2b, CCR3, and CCR1. In a preferred embodiment, the target region is a polypurine or homopurine site within the coding region of the human CCR5 gene. Three homopurine sites in the coding region of the CCR5 gene that are especially useful as target sites for triplex-forming molecules are from positions 509-518, 679-690 and 900-908 relative to the ATG start codon.

The homopurine site from 679-690 partially encompasses the site of the nonsense mutation created by the Δ32 mutation. PNAs that bind to this target site are particularly useful. One PNA that binds to this target sequence is designated P-679 and is represented by the following sequence: JTJTTJTTJT-e-e-e-TCTTCTTCTC-Lys-Lys-Lys, where J=pseudoisocytosine and e=flexible linker (SEQ ID NO:7). The flexible linker molecules can be 8-amino-3,6-dioxaoctanoic acid, 6-aminohexanoic acid, or poly(ethylene) glycol monomers. This dimeric bis-PNA contains two linked PNA segments and is designed to form a PNA/DNA/PNA triplex clamp on the purine-rich DNA strand of the 674 site (specifically at position 679). The ability of bis-PNAs to form such clamp structures at chromosomal targets inside cells has been previously demonstrated. The examples below using gel mobility shift assays to test the affinity of PNA-679 to its binding site in the CCR5 gene in vitro, which revealed strong binding by this molecule to its target site in the CCR5 gene. The examples below using allele-specific PCR also demonstrate that PNA-679 (in combination with a DNA donor containing a nonsense mutation) can induce mutation of the endogenous CCR5 gene in the human monocytic acute leukemia cell line, THP-1.

Additional exemplary PNAs that can bind to the human CCR5 gene include, but are not limited to, the bis-PNA represented by the following sequence: Lys-Lys-Lys-JTJTTJTTJT-e-e-e-TCTTCTTCTC-Lys-Lys-Lys (679-688) where J=pseudoisocytosine and e=flexible linker (SEQ ID NO:8), and the PNA represented by the following sequence: CTTGTCATGG-Lys-Lys-Lys (522-531) (SEQ ID NO:9). The numbers in parentheses indicate the sites that the PNAs bind to relative to the start ATG.

D. Donor Oligonucleotides

The triplex forming oligonucleotides or peptide nucleic acids may be administered in combination with, or tethered to a donor oligonucleotide via a mixed sequence linker or used in conjunction with a non-tethered donor oligonucleotide that is homologous to the target sequence. Donor oligonucleotides are also referred to herein as donor fragments, donor nucleic acids, donor DNA, or donor DNA fragments. This strategy is intended to exploit the ability of a triplex, itself, to provoke DNA repair, potentially increasing the probability of recombination with the homologous donor DNA. It is understood in the art that a greater number of homologous positions within the donor fragment will increase the probability that the donor fragment will be recombined into the target sequence, target region, or target site. Tethering of a donor oligonucleotide to a triplex-forming molecule facilitates target site recognition via triple helix formation while at the same time positioning the tethered donor fragment for possible recombination and information transfer. Triplex-forming molecules also effectively induce homologous recombination of non-tethered donor oligonucleotides. The term “recombinagenic” as used herein, is used to define a DNA fragment, oligonucleotide, peptide nucleic acid, or composition as being able to recombine into a target site or sequence or induce recombination of another DNA fragment, oligonucleotide, or composition.

Non-tethered, or unlinked fragments may range in length from 20 nucleotides to several thousand. It is to be understood that the donor oligonucleotide molecules, whether linked or unlinked, can exist in single stranded or double stranded form. It is to be understood that the donor fragment to be recombined can be linked or un-linked to the triplex forming oligonucleotide. The linked donor fragment may range in length from 4 nucleotides to 100 nucleotides, preferably from 4 to 50 nucleotides in length. However, the unlinked donor fragments have a much broader range: from 20 nucleotides to several thousand. It is preferable that the triplex forming recombinagenic oligonucleotide is at least 10 nucleotides in length. It is more preferable that the oligonucleotide be at least 20 nucleotides in length.

The donor oligonucleotides contain at least one mutated, inserted or deleted nucleotide relative to the target DNA sequence. The target sequence is preferably within or is adjacent to a portion of a HIV receptor gene important to its function in HIV entry into cells, such as sequences that are involved in efficient expression of the receptor, transport of the receptor to the cell surface, stability of the receptor, viral binding by the receptor, or endocytosis of the receptor. Target sequences can be within the coding DNA sequence of the gene or within introns. Target sequences can also be within DNA sequences which regulate expression of the target gene, including promoter or enhancer sequences.

The donor oligonucleotides can contain a variety of mutations relative to the target sequence. Representative types of mutations include, but are not limited to point mutations, deletions and insertions. Point mutations can cause missense or nonsense mutations. Deletions and insertions can result in frameshift mutations or deletions. Such mutations can cause one or more deficiencies in the ability of the cell surface HIV receptor to bind to HIV and allow its transport into the cell. The ultimate effect of the mutation in or adjacent to the target sequence is to inhibit or reduce the ability of the cell surface HIV receptor to bind to viral particles and permit entry of the viral particles into the cell.

E. Methods for Determining Triplex Formation

The preferred conditions under which a triple-stranded structure will form are standard assay conditions for in vitro mutagenesis and physiological conditions for in vivo mutagenesis. (See for example, Moser and Dervan, Science 238:645 (1987); Praseuth et al., Proc. Natl. Acad. Sci. USA 85:1349 (1988); Mergny et al., Biochemistry 30:9791 (1991); Beal and Dervan, Science 251:1360 (1991); Mergny et al., Biochemistry 30:9791 (1991) and Beal and Dervan, Nuc. Acids Res. 11:2773 (1992).

A useful measure of triple helix formation is the equilibrium dissociation constant, K_{d}, of the triplex, which can be estimated as the concentration of triplex-forming molecule at which triplex formation is half-maximal. Preferably, the triplex-forming molecule has a binding affinity for the target sequence in the range of physiologic interactions. The preferred binding affinity is a K_{d} less than or equal to approximately 10⁻⁷ M. Most preferably, the K_{d} is less than or equal to 2×10⁻⁸ M in order to achieve significant intramolecular interactions.

A variety of methods are available to determine the K_{d} of an oligonucleotide/target pair. In the Example that follows, the K_{d} was estimated using a gel mobility shift assay (R. H. Durland et al., Biochemistry 30, 9246 (1991)). In the example below using this method, as bis-peptide nucleic acid (PNA) corresponding to the sequence located at position 674 (relative tio the ATG start codon) of the human CCR5 gene was annealed to make a PNA/DNA/PNA triplex clamp on the purine-rich strand of the target site. The annealed oligonucleotide was incubated overnight (approximately 18-24 hours) at 37° C. with increasing concentrations of the bis-PNA and 2 μg of plasmid containing the PNA binding site flanked by known restriction enzyme sites. The reactions were then subjected to restriction enzymes and then to gel electrophoresis in a 10% non-denaturing polyacrylamide (19:1 acrylamide:bisacrylamide) gel containing 89 mM Tris, 89 mM boric acid, pH 7.2, and 10 mM MgCl₂ (for pH 7.2 conditions) using a BioRad Mini PROTEAN 3 apparatus for ˜4 hours at 65V. The gels were then stained with silver stain for visualization. The dissociation constant (K_{d}) can be determined as the concentration of bis-PNA in which half was bound to the target sequence and half was unbound.

F. Cell Targeting Moieties and Protein Transduction Domains

Formulations of the triplex-forming molecules embrace fusions of the triplex-forming molecules or modifications of the triplex-forming molecules, wherein the triplex-forming molecules are fused to another moiety or moieties. Such analogs may exhibit improved properties such as increased cell membrane permeability, activity and/or stability. Examples of moieties which may be linked or unlinked to the oligonucleotides include, for example, targeting moieties which provide for the delivery of oligonucleotides to specific cells, e.g., antibodies to hematopoeitic stem cells, CD34⁺ cells, T cells or any other preferred cell type, as well as receptor and ligands expressed on the preferred cell type. Preferably, the moieties target hematopoeitic stem cells. Other moieties that may be provided with the oligonucleotides include protein transduction domains (PTDs), which are short basic peptide sequences present in many cellular and viral proteins that mediate translocation across cellular membranes. Example protein transduction domains that are well-known in the art include the Antennapedia PTD and the TAT (transactivator of transcription) PTD.

G. Additional Mutagenic Agents

The triplex-forming molecules disclosed herein can be used alone or in combination with other mutagenic agents. As used herein, two agents are said to be used in combination when the two agents are co-administered, or when the two agents are administered in a fashion so that both agents are present within the cell or serum simultaneously. In a preferred embodiment, the additional mutagenic agents are conjugated or linked to the triplex-forming molecule. Additional mutagenic agents that can be used in combination with triplex-forming molecules include agents that are capable of directing mutagenesis, or are nucleic acid crosslinkers, or are radioactive agents, or are alkylating groups, or are molecules that can recruit DNA-damaging cellular enzymes. Other suitable mutagenic agents include, but are not limited to, chemical mutagenic agents such as alkylating, bialkylating or intercalating agents. A preferred agent for co-administration is psoralen-linked oligonucleotides as described in PCT/US/94/07234 by Yale University.

H. Additional Prophylactic or Therapeutic Agents

The triplex-forming molecules disclosed herein can be used alone or in combination with other prophylactic or therapeutic agents. As used herein, two agents are said to be used in combination when the two agents are co-administered, or when the two agents are administered in a fashion so that both agents are present within the cell or serum simultaneously. Suitable additional prophylactic or therapeutic agents include those useful to treat or prevent HIV infection. Suitable therapeutic agents include those typically used for “HAART”, which is an acronym for highly active antiretroviral therapy for the treatment of HIV-1 infection. HAART therapy typically encompasses a double nucleoside (NRTI) backbone plus either a non-nucleoside reverse transcriptase inhibitor (NNRTI) or a ritonavir pharmacologically enhanced protease inhibitor (PI/r). However the actual therapeutic composition in terms of both class and active agent varies depending upon availability of each agent and a patient's individual tolerance for each ingredient, among others. Accordingly, use of the term “HAART” is meant to broadly encompass all combinations of active therapeutic agents that the art would ascribe to this term. Exemplary HAART therapeutic agents include nucleoside & nucleotide reverse transcriptase inhibitors (NRTI), non-nucleoside reverse transcriptase inhibitors (nNRTI), protease inhibitors, integrase inhibitors, entry inhibitors and maturation inhibitors.

Suitable entry inhibitors include other therapeutic agents that function as antagonists to HIV cell surface receptors, including CCR5. CCR5 antagonists include small molecule noncompetitive allosteric antagonists which bind in a cavity formed between several transmembrane helices of the CCR5 protein, including, but not limited to, TAK-779, TAK-220, TAK-652, aplaviroc, maraviroc and vicroviroc.

### II. Methods of Use

A. Inactivation of Cell Surface Receptors for HIV

Triplex-forming molecules bind/hybridize to a target sequence within or adjacent to a human gene encoding a cell surface receptor for HIV, forming a triplex structure. The binding of the triple-forming molecule to the target region stimulates mutations within or adjacent to the target region using cellular DNA synthesis, recombination, and repair mechanisms. In targeted recombination, a triplex forming molecule is administered to a cell in combination with a separate donor oligonucleotide fragment which minimally contains a sequence substantially complementary to the target region or a region adjacent to the target region, referred to herein as the donor fragment. The donor fragment can further contain nucleic acid sequences which are to be inserted within the target region. The co-administration of a triplex forming oligonucleotide with the fragment to be recombined increases the frequency of insertion of the donor fragment within the target region when compared to procedures which do not employ a triplex forming oligonucleotide.

The triplex-forming molecules in combination with the donor oligonucleotides induce site-specific mutations or alterations of the nucleic acid sequence within or adjacent to the target sequence. The target sequence is preferably within or is adjacent to a portion of a HIV receptor gene important to its function in HIV entry into cells, such as sequences that are involved in efficient expression of the receptor, transport of the receptor to the cell surface, stability of the receptor, viral binding by the receptor, or endocytosis of the receptor. Target sequences can be within the coding DNA sequence of the gene or within introns. Target sequences can also be within DNA sequences which regulate expression of the target gene, including promoter or enhancer sequences.

The triplex-forming molecules in conjunction with donor oligonucleotides can induce any of a range of mutations in or adjacent to the target sequence. Representative types of mutations include, but are not limited to point mutations, deletions and insertions. Point mutations can cause missense or nonsense mutations. Deletions and insertions can result in frameshift mutations or deletions. Such mutations can cause one or more deficiencies in the ability of the cell surface HIV receptor to bind to HIV and allow its transport into the cell. For example, mutations can result in reduced expression (transcription and/or translation) of the target gene. Mutations can also result in a defect in the transport of the receptor to the cell surface or a reduction in the stability of the protein such that its presentation at the cell surface is reduced or inhibited. Mutations can also reduce the ability of the receptor to be internalized by endocytosis, or to be routed through proper endocytic pathways. Mutations can also reduce or inhibitor binding of HIV viral particles by the cell surface receptor.

The ultimate effect of the mutation in or adjacent to the target sequence is to inhibit or reduce the ability of the cell surface HIV receptor to bind to viral particles and permit entry of the viral particles into the cell. The particular HIV cell surface receptor gene targeted by the triplex-forming molecule determines which strains of HIV will display reduced or inhibited binding and entry into the cell. HIV-1 isolates exhibit marked differences in their ability to infect CD4⁺T cells. While all strains infect primary CD4⁺T cells, most primary isolates also infect macrophages (M tropic) but fail to infect transformed CD4⁺T cell lines. Other isolates replicate well in CD4⁺T cell lines (T tropic) but fail to infect macrophages. The underlying source of permissiveness for M and T tropic viruses is determined by the co-receptor used by the HIV strains. CCR5 confers susceptibility to infection by certain M-tropic (R5-tropic) strains of HIV-1, whereas CXCR4, serves as a cofactor for T tropic (X4-tropic) HIV-1 strains. Thus, mutations in the CCR5 gene can create cells that are R5-tropic virus-resistant cells, and mutations in the CXCR4 gene can create cells that are X4-tropic virus-resistant cells. In some embodiments, more than one species of triplex-forming molecule is used to induce mutations in more than one cell surface HIV receptor. This can result in cells that are resistant to HIV strains with more than one tropism.

In one embodiment, the compositions and methods disclosed herein are used to cause mutations in the human CCR5 gene. In a preferred embodiment, the mutation mimics a naturally occurring polymorphism in the human CCR5 gene that causes a 32 basepair deletion of the CCR5 receptor referred to commonly in the art as the CCR5 Δ32 mutation. This mutation causes a frameshift and deletion of the last three transmembrane domains of the CCR5 protein.

B. Generation of HIV Receptor Mutant Cell Lines

The triplex-forming molecules disclosed herein are useful for the generation of cell lines containing a diverse range of mutations in genes encoding cell surface HIV receptors. Cell lines can contain mutations in or adjacent to one or more genes encoding cell surface receptors and/or can contain one or more mutations in or adjacent to a single gene encoding a cell surface receptor for HIV. Such cell lines are useful for the screening and development of other HIV therapeutic agents, including other agents that inhibit or reduce the entry of HIV into a cell. Any cell that expresses at least one cell surface receptor for HIV and that is capable of being transfected or transduced with a triplex-forming molecule can be used, including primary isolated cells and immortalized cell lines. The cells are preferably hematopoietic in origin and can be hematopoietic stem cells. Other suitable hematopoietic cells include T cells. T cells include all cells which express CD3, including T cell subsets which also express CD4 and CD8. T cells include both naive and memory cells and effector cells such as CTL. T-cells also include regulatory cells such as Th1, Tc1, Th2, Tc2, Th3, Treg, and Tr1 cells. T cells used for generation of cell lines containing mutations in genes encoding cell surface HIV receptors are preferably CD4⁺ T cells.

C. Treatment of Subjects with or at Risk of Developing an HIV Infection

In general, the compositions and methods described herein are useful for treating a subject having or being predisposed to HIV infection. The compositions are useful as prophylactic compositions, which confer resistance in a subject to HIV. The compositions are also useful as therapeutic compositions, which can be used to initiate or enhance a subject's resistance to HIV infection. The compositions and methods generate CD4⁺ immune cells which are resistant to infection by HIV by altering the expression, localization, stability, binding activity and/or endocytosis of at least one cell surface receptor for HIV. The result of treatment with the compositions and methods disclosed herein is to prevent infection of an individual with HIV or to reduce the viral load in a subject that is already infected with HIV. Another result of treatment can be an increase in CD4 counts in subjects infected with HIV. Methods for assessing HIV viral load and CD4 counts are well known in the art.

Preferably, the compositions and methods described herein can be used to treat or prevent any disease or condition that arises from HIV infection, such as AIDS and ARC. It should be recognized that the methods disclosed herein can be practiced in conjunction with existing antiviral therapies to effectively treat or prevent HIV infection and diseases and conditions that arise from HIV infection.

i. Ex Vivo Gene Therapy for Treating or Preventing HIV Infection

In one embodiment, ex vivo gene therapy of cells is used for the treatment or prevention of HIV infection in a subject. For ex vivo prophylaxis or therapy of HIV infection, cells are isolated from a subject and contacted ex vivo with the compositions disclosed herein to produce cells containing mutations in or adjacent to genes encoding HIV cell surface receptors including, but not limited to, CXCR4, CCR5, CCR2b, CCR3, and CCR1. In a preferred embodiment, the cells are isolated from the subject to be treated or from a syngenic host. Target cells are removed from a subject prior to contacting with triplex-forming molecules and donor oligonucleotides. The cells can be hematopoietic progenitor or stem cells. In a preferred embodiment, the target cells are CD34⁺ hematopoietic stem cells. CD34⁺ hematopoietic stem cells have been shown to be resistant to HIV infection. The resistance of CD34⁺ cells to HIV infection makes them an especially attractive cell type for gene therapy of HIV using the compositions and methods disclosed herein because they can be taken from HIV infected individuals and mutated without fear of HIV contamination.

Such stem cells can be isolated and enriched by one of skill in the art. Methods for such isolation and enrichment of CD34⁺ and other cells are known in the art and disclosed for example in U.S. Pat. Nos. 4,965,204; 4,714,680; 5,061,620; 5,643,741; 5,677,136; 5,716,827; 5,750,397 and 5,759,793. As used herein in the context of compositions enriched in hematopoietic progenitor and stem cells, “enriched” indicates a proportion of a desirable element (e.g. hematopoietic progenitor and stem cells) which is higher than that found in the natural source of the cells. A composition of cells may be enriched over a natural source of the cells by at least one order of magnitude, preferably two or three orders, and more preferably 10, 100, 200 or 1000 orders of magnitude.

In humans, CD34⁺ cells can be recovered from cord blood, bone marrow or from blood after cytokine mobilization effected by injecting the donor with hematopoietic growth factors such as granulocyte colony stimulating factor (G-CSF), granulocyte-monocyte colony stimulating factor (GM-CSF), stem cell factor (SCF) subcutaneously or intravenously in amounts sufficient to cause movement of hematopoietic stem cells from the bone marrow space into the peripheral circulation. Initially, bone marrow cells may be obtained from any suitable source of bone marrow, e.g. tibiae, femora, spine, and other bone cavities. For isolation of bone marrow, an appropriate solution may be used to flush the bone, which solution will be a balanced salt solution, conveniently supplemented with fetal calf serum or other naturally occurring factors, in conjunction with an acceptable buffer at low concentration, generally from about 5 to 25 mM. Convenient buffers include Hepes, phosphate buffers, lactate buffers, etc.

Cell scan be selected by positive and negative selection techniques. Cells can be selected using commercially available antibodies which bind to hematopoietic progenitor or stem cell surface antigens, e.g. CD34, using methods known to those of skill in the art. For example, the antibodies may be conjugated to magnetic beads and immunogenic procedures utilized to recover the desired cell type. Other techniques involve the use of fluorescence activated cell sorting (FACS). The CD34 antigen, which is found on progenitor cells within the hematopoietic system of non-leukemic individuals, is expressed on a population of cells recognized by the monoclonal antibody My-10 (i.e., express the CD34 antigen) and can be used to isolate stem cell for bone marrow transplantation. My-10 has been deposited with the American Type Culture Collection (Rockville, Md.) as HB-8483 is commercially available as anti-HPOA 1. Additionally, negative selection of differentiated and “dedicated” cells from human bone marrow can be utilized, to select against substantially any desired cell marker. For example, progenitor or stem cells, most preferably CD34⁺ cells, can be characterized as being any of CD3⁻, CD7⁻, CD8⁻, CD10⁻, CD14⁻, CD15⁻, CD19⁻, CD20⁻, CD33⁻, Class II HLA⁺ and Thy-1⁺.

Once progenitor or stem cells have been isolated, they may be propagated by growing in any suitable medium. For example, progenitor or stem cells can be grown in conditioned medium from stromal cells, such as those that can be obtained from bone marrow or liver associated with the secretion of factors, or in medium comprising cell surface factors supporting the proliferation of stem cells. Stromal cells may be freed of hematopoietic cells employing appropriate monoclonal antibodies for removal of the undesired cells.

The isolated cells are contacted ex vivo with a combination of triplex-forming molecules and donor oligonucleotides in amounts effective to cause the desired mutations in or adjacent to genes encoding cell surface receptors for HIV. These cells are referred to herein as modified cells. Methods for transfection of cells with oligonucleotides and peptide nucleic acids are well known in the art (Koppelhus, et al., Adv. Drug Deliv. Rev., 55(2): 267-280 (2003)).

The modified cells can be maintained or expanded in culture prior to administration to a subject. Culture conditions are generally known in the art depending on the cell type. Conditions for the maintenance of CD34⁺ in particular have been well studied, and several suitable methods are available. A common approach to ex vivo multi-potential hematopoietic cell expansion is to culture purified progenitor or stem cells in the presence of early-acting cytokines such as interleukin-3. It has also been shown that inclusion, in a nutritive medium for maintaining hematopoietic progenitor cells ex vivo, of a combination of thrombopoietin (TPO), stem cell factor (SCF), and flt3 ligand (Flt-3L; i.e., the ligand of the flt3 gene product) was useful for expanding primitive (i.e., relatively non-differentiated) human hematopoietic progenitor cells in vitro, and that those cells were capable of engraftment in SCID-hu mice (Luens et al., 1998, Blood 91:1206-1215). In other known methods, cells can be maintained ex vivo in a nutritive medium (e.g., for minutes, hours, or 3, 6, 9, 13, or more days) comprising murine prolactin-like protein E (mPLP-E) or murine prolactin-like protein F (mPIP-F; collectively mPLP-E/IF) (U.S. Pat. No. 6,261,841). It will be appreciated that other suitable cell culture and expansion method can be used in accordance with the invention as well. Cells can also be grown in serum-free medium, as described in U.S. Pat. No. 5,945,337.

In another embodiment, the modified hematopoietic stem cells are differentiated in ex vivo into CD4⁺ cells culture using specific combinations of interleukins and growth factors prior to administration to a subject using methods well known in the art. The cells may be expanded ex vivo in large numbers, preferably at least a 5-fold, more preferably at least a 10-fold and even more preferably at least a 20-fold expansion of cells compared to the original population of isolated hematopoietic stem cells.

In another embodiment cells for ex vivo gene therapy, the cells to be used can be dedifferentiated somatic cells. Somatic cells can be reprogrammed to become pluripotent stem-like cells that can be induced to become hematopoietic progenitor cells. The hematopoietic progenitor cells can then be treated with triplex-forming molecules and donor oligonucleotides as described above with respect to CD34⁺ cells to produce recombinant immune cells that do not express functional receptors involved in HIV infection. Representative somatic cells that can be reprogrammed include, but are not limited to fibroblasts, adipocytes, and muscles cells. Hematopoietic progenitor cells from induced stem-like cells have been successfully developed in the mouse (Hanna, J. et al. Science, 318:1920-1923 (2007)).

To produce hematopoietic progenitor cells from induced stem-like cells, somatic cells are harvested from a host. In a preferred embodiment, the somatic cells are autologous fibroblasts. The cells are cultured and transduced with vectors encoding Oct4, Sox2, Klf4, and c-Myc transcription factors. The transduced cells are cultured and screened for embryonic stem cell (ES) morphology and ES cell markers including, but not limited to AP, SSEA1, and Nanog. The transduced ES cells are cultured and induced to produce induced stem-like cells. Cells are then screened for CD41 and c-kit markers (early hematopoietic progenitor markers) as well as markers for myeloid and erythroid differentiation.

The modified hematopoietic stem cells, modified differentiated CD4⁺ cells or modified induced hematopoietic progenitor cells are then introduced into a subject. Delivery of the cells may be effected using various methods and includes most preferably intravenous administration by infusion as well as direct depot injection into periosteal, bone marrow and/or subcutaneous sites.

The subject receiving the modified cells may be treated for bone marrow conditioning to enhance engraftment of the cells. The recipient may be treated to enhance engraftment, using a radiation or chemotherapeutic treatment prior to the administration of the cells. Upon administration, the cells will generally require a period of time to engraft. Achieving significant engraftment of hematopoietic stem or progenitor cells typically takes a period week to months.

A high percentage of engraftment of modified hematopoietic stem cells or modified differentiated CD4⁺ cells is not envisioned to be necessary to achieve significant prophylactic or therapeutic effect. The examples below demonstrate that mutations introduced into genes using the compositions and methods described herein are stable over long periods of time. It is expected that the engrafted cells will expand over time following engraftment to increase the percentage of modified cells. The modified cells are resistant to infection by HIV relative to unmodified cells due to altered expression, localization, stability, binding activity and/or endocytosis of at least one cell surface receptor for HIV. Therefore, in a subject with an HIV infection, the modified cells are expected to have a competitive advantage over non-modified cells. It is expected that engraftment of only a small number or small percentage of modified hematopoietic stem cells will be required to provide a prophylactic or therapeutic effect.

In preferred embodiments, the cells to be administered to a subject will be autologous, e.g. derived from the subject, or syngenic. Nevertheless, allogeneic cell transplants are also envisioned, and allogeneic bone marrow transplants are carried out routinely. Allogeneic cell transplantation can be offered to those patients who lack an appropriate sibling donor by using bone marrow from antigenically matched, genetically unrelated donors (identified through a national registry), or by using hematopoietic progenitor or stem-cells obtained or derived from a genetically related sibling or parent whose transplantation antigens differ by one to three of six human leukocyte antigens from those of the patient.

ii. In Vivo Gene Therapy for Treating or Preventing HIV Infection

In another embodiment, the triplex-forming molecules are administered directly to a subject with or having been predisposed to HIV infection. In general, methods of administering oligonucleotides and related molecules are well known in the art. Any acceptable method known to one of ordinary skill in the art may be used to administer a formulation to the subject. The administration may be localized (i.e., to a particular region, physiological system, tissue, organ, or cell type) or systemic. Triplex-forming molecules and donor oligonucleotides can be administered by a number of routes including, but not limited to: oral, inhalation (nasal or pulmonary), intravenous, intraperitoneal, intramuscular, transdermal, subcutaneous, topical, sublingual, or rectal means. Injections can be e.g., intravenous, intradermal, subcutaneous, intramuscular, or intraperitoneal. In some embodiments, the injections can be given at multiple locations. The compositions can also be administered directly to the bone marrow or to an appropriate lymphoid tissue, such as the spleen, lymph nodes or mucosal-associated lymphoid tissue.

Formulations

The compositions are preferably employed for therapeutic uses in combination with a suitable pharmaceutical carrier. Such compositions comprise an effective amount of the compound, and a pharmaceutically acceptable carrier or excipient. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids some of which are.

It is understood by one of ordinary skill in the art that nucleotides administered in vivo are taken up and distributed to cells and tissues (Huang, et al., FEBS Lett. 558(1-3):69-73 (2004)). For example, Nyce et al. have shown that antisense oligodeoxynucleotides (ODNs) when inhaled bind to endogenous surfactant (a lipid produced by lung cells) and are taken up by lung cells without a need for additional carrier lipids (Nyce and Metzger, Nature, 385:721-725 (1997). Small nucleic acids are readily taken up into T24 bladder carcinoma tissue culture cells (Ma, et al., Antisense Nucleic Acid Drug Dev. 8:415-426 (1998).

The compounds may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles or microspheres formed of biodegradable or non-biodegradable polymers or proteins or liposomes for targeting to cells. Such systems are well known to those skilled in the art and may be optimized for use with the appropriate nucleic acid.

Various methods for nucleic acid delivery are described, for example, in Sambrook et al., 1989, Molecular Cloning: A Laboratory Manual, Cold Spring Harbor Laboratory, New York; and Ausubel et al., 1994, Current Protocols in Molecular Biology, John Wiley & Sons, New York. Such nucleic acid delivery systems comprise the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. The nucleic acid delivery system can be provided to the cell by endocytosis, receptor targeting, coupling with native or synthetic cell membrane fragments, physical means such as electroporation, combining the nucleic acid delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, with an added preservative. The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). Preservatives and other additives may also be present such as, for example, antimicrobials, antioxidants, chelating agents and inert gases. In addition, sterile, fixed oils are conventionally employed as a solvent or suspending medium. For this purpose any bland fixed oil including synthetic mono- or di-glycerides may be employed. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The compound alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

In some embodiments, the compound described above may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the compounds are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett. 14(19):4975-4977 (2004)) and in vivo (Soutschek, et al., Nature 432(7014):173-178 (2004)). In addition, it has been shown that binding of steroid conjugated oligonucleotides to different lipoproteins in the bloodstream, such as LDL, protect integrity and facilitate biodistribution (Rump, et al., Biochem. Pharmacol. 59(11):1407-1416 (2000)). Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also describes methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

Methods of Administration

In general, methods of administering compounds, including oligonucleotides, peptide nucleic acids and related molecules, are well known in the art. In particular, the routes of administration already in use for nucleic acid therapeutics, along with formulations in current use, provide preferred routes of administration and formulation for the oligonucleotides described above. Preferably the oligonucleotides are injected into the organism undergoing genetic manipulation, such as a human requiring gene therapy for the treatment or prevention of HIV infection.

Compositions can be administered by a number of routes including, but not limited to: oral, intravenous, intraperitoneal, intramuscular, transdermal, subcutaneous, topical, sublingual, or rectal means. The preferred route of administration is intravenous. Compounds can also be administered via liposomes. Such administration routes and appropriate formulations are generally known to those of skill in the art.

Administration of the formulations may be accomplished by any acceptable method which allows the triplex-forming oligonucleotide and optionally a donor nucleotide, to reach its target.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The oligonucleotides may be delivered in a manner which enables tissue-specific uptake of the agent and/or nucleotide delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the triplex-forming oligonucleotides, and optionally donor oligonucleotides, over a certain time period, for example, hours, days, weeks, months or years. This may be accomplished, for example, by repeated administrations of a formulation or by a sustained or controlled release delivery system in which the oliogonucleotides are delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which the miRNA is contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the oligonucleotides. In addition, a pump-based hardware delivery system may be used to deliver one or more embodiments.

Examples of systems in which release occurs in bursts include systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Unless defined otherwise, all technical and scientific terms used herein have the same meanings as commonly understood by one of skill in the art to which the disclosed invention belongs. Publications cited herein and the materials for which they are cited are specifically incorporated by reference.

### EXAMPLES

The present invention may be further understood by reference to the following non-limiting examples.

### Example 1

### Binding of a Peptide Nucleic Acid to a Fragment of the CCR5 Gene and Design of Donor Oligonuclotides

A bis-PNA was designed to bind to CCR5 with high affinity and specificity. The PNA is represented by the following sequence: JTJTIITTJT-e-e-e-TCTICTTCTC-Lys-Lys-Lys, where J=pseudoisocytosine and e=flexible linker (SEQ ID NO:7). In vitro binding was evaluated using a gel-shift assay which confirmed that the molecule binds to the target site at μM concentrations. Three single-stranded 60mer donor oligonucleotides were designed and synthesized. All donors were completely homologous to the template strand (antisense) of the CCR5 gene, except for the 6 center bases which contain a stop codon. The donors were designed to place this stop codon near the Δ32 mutation site to mimic this mutation which has been shown to inactivate CCR5 by truncation and mislocalization of the protein. One donor (983) was designed to introduce a 6 bp DdeI restriction site (which also contains an inframe stop codon). The two other donors (980 and 987) were designed to mutate the 6 bases immediately adjacent (either 5′ or 3′) to the first 6 bp site and will be used for the creation of comp hets. All 6 bp mutations were site-directed into a plasmid containing full-length CCR5 and will be used as controls for allele-specific PCR (ASPCR).

### Example 2

### Mutation of the CCR5 Gene in THP-1 Human Leukemia Cells Using PNAs and Donor Oligonucleotides

ASPCR primers were designed for specific amplification of the mutant CCR5 sequence. Allele-specific forward primers were designed containing the specific 6 bp mutant sequence at its 3′ end. When paired with the gene-specific reverse primer, the allele-specific primer will preferentially amplify the mutant CCR5 gene. To determine ASPCR conditions, gradients were run with plasmid controls to determine the optimal Tm for specific amplification of the mutant sequence. Combinations of donor molecules and bis-PNA were then transfected into THP-1 cells to test for homologous recombination, and the genomic DNA from these cells was analyzed by ASPCR.

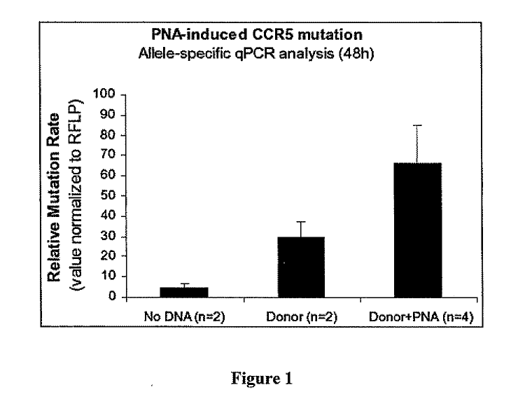

THP-1 cells were transfected with either nothing, 5 μg of 983 donor, or 5 μg of 983 donor with 2 μM PNA. 48 hours after transfection an aliquot of approximately one million cells were taken and genomic DNA was isolated. Allele-specific PCR was performed on DNA samples using 983 allele-specific primers and a band corresponding to the UP mutation is seen in the treated samples. Plasmid DNA containing either wild-type or 6 bp 983 mutant sequence were also run as a PCR control. Samples were run on a 1% agarose gel. The sample genomic DNA samples were assayed using the real-time ASPCR assay with normalization to gene specific primers. ASPCR of the genomic DNA from these transfections showed specific amplification corresponding to the mutant DNA (FIG. 1). Low background amplification can also be seen at this annealing temperature and cycle threshold, and plasmid controls confirm specific amplification of the mutant sequence.

ASPCR of genomic DNA harvested from cells treated with either the 980 or 987 donor demonstrate the specificity of this assay even when the mutations are adjacent to each other. THP-1 cells were transfected with either nothing, 5 μg of 980 donor or 5 μg or 987 donor. 48 hours after transfection an aliquot of approximately one million cells were taken and genomic DNA was isolated. ASPCR was performed using either 980 or 987 specific primers. The PCR reaction was run on a 1% agarose gel. No amplification was seen with the 980-specific primers when 987 donor was used or with the 987-primers when the 980 donor was used.

Persistence of the 983 mutation at has also been detected at 500 hrs at the DNA level and at 220 hrs at the RNA level. THP-1 cells were transfected with 5 μg of 983 donor or nothing and aliquots were taken at the given times. Genomic DNA was isolated and ASPCR was performed and analyzed on a 1% agarose gel.

Those skilled in the art will recognize, or be able to ascertain using no more than routine experimentation, many equivalents to the specific embodiments of the invention described herein. Such equivalents are intended to be encompassed by the following claims.

We claim:

1. A recombinagenic or mutagenic composition comprising
a triplex-forming molecule that binds to duplex DNA at in a sequence-specific manner to form a triple-stranded structure, and
a donor oligonucleotide,
wherein the triplex-forming molecule binds to a target site in or adjacent to a human gene that encodes a cell surface receptor for HIV.

2. The recombinagenic or mutagenic composition of claim 1 wherein the triplex-forming molecule is selected from the group consisting of triplex-forming oligonucleotides and peptide nucleic acids.

3. The recombinagenic or mutagenic composition of claim 2 wherein the peptide nucleic acid is a two-stranded bis-peptide nucleic acid.

4. The recombinagenic or mutagenic composition of claim 2 wherein the triplex-forming oligonucleotide comprises one or more chemically modified oligonucleotides.

5. The recombinagenic or mutagenic composition of claim 1 wherein the target site is in or within a human chemokine gene.

6. The recombinagenic or mutagenic composition of claim 5 wherein the human chemokine gene is selected from the group consisting of CXCR4, CCR5, CCR2b, CCR3, and CCR1.

7. The recombinagenic or mutagenic composition of claim 5 wherein the human chemokine gene is CCR5.

8. The recombinagenic or mutagenic composition of claim 7 wherein the target site encompasses the site of the Δ32 mutation in the CCR5 gene.

9. The recombinagenic or mutagenic composition of claim 1 wherein the donor oligonucleotide comprises one or more nucleotide mutations, deletions or insertions relative to the target duplex DNA nucleotide sequence.

10. The recombinagenic or mutagenic composition of claim 9 wherein the donor oligonucleotide comprises one or more point mutations that cause missense or nonsense mutations in the target duplex DNA nucleotide sequence wherein the missense or nonsense mutations result in a frameshift or deletion in the target duplex DNA.

11. The recombinagenic or mutagenic composition of claim 9 wherein the mutation, deletion or insertion results in a deficiency in a cell surface receptor for HIV selected from the group consisting of reduced expression of the receptor, defects in transport of the receptor to the cell surface, reduced stability of the receptor protein, reduced binding of HIV by the receptor and defects in endocytosis of the receptor.

12. A method for targeted recombination or mutation of a gene encoding a cell surface receptor for HIV comprising contacting cells with the composition of claim 1 wherein the donor oligonucleotide comprises one or more nucleotide mutations, deletions or insertions relative to the target duplex DNA nucleotide sequence.

13. A method for prophylaxis or treatment of HIV infection in subjects with or at risk of developing an HIV infection comprising
a) isolating cells from a host,
b) contacting the cells ex vivo with the composition of claim 1 wherein the donor oligonucleotide comprises one or more nucleotide mutations, deletions or insertions relative to the target duplex DNA nucleotide sequence,
c) expanding the cells in culture, and
d) administering the cells to a subject in need thereof.

14. The method of claim 13 wherein the cells are resistant to infection by one or more strains of HIV.

15. The method of claim 14 wherein the cells are resistant to R5-trophic HIV strains.

16. The method of claim 13 wherein the cells are isolated from the subject to be treated or a syngenic host.

17. The method of claim 13 wherein the cells are CD34⁺ cells.

18. The method of claim 15 further comprising differentiating the cells into CD4⁺ cells prior to step d).

19. A method for prophylaxis or treatment of HIV infection in subjects with or at risk of developing an HIV infection comprising administering to a subject in need thereof the composition of claim 1 wherein the donor oligonucleotide comprises one or more nucleotide mutations, deletions or insertions relative to the target duplex DNA nucleotide sequence.

20. Cell lines generated by the method of claim 12.
